(19) United States
(12) Patent Application Publication
(10) Pub. No.: US 2026/0152587 A1
(43) Pub. Date: Jun. 4, 2026

(54) POLYDIACETYLENE-BASED COMPOUND, ITS COMPOSITION AND CHEMICAL SENSORS FOR DETECTING CYANIDE IONS

(71) Applicant: PUKYONG NATIONAL UNIVERSITY INDUSTRY-UNIVERSITY COOPERATION FOUNDATION, Busan (KR)

(72) Inventors: Songyi LEE, Busan (KR); Seongman LEE, Gyeongsangbuk-do (KR)

(21) Appl. No.: 19/177,326

(22) Filed: Apr. 11, 2025

(30) Foreign Application Priority Data
May 31, 2024 (KR) 10-2024-0071648

(51) Int. Cl. C08F 38/02; C08F 2/48; G01N 21/77; G01N 21/78

(52) U.S. Cl. CPC C08F 38/02; C08F 2/48; G01N 21/78; G01N 2021/7786

(57) ABSTRACT

The present disclosure relates to a polydiacetylene-based compound for detecting cyanide ions, a composition including the same, and a chemical sensor, and more particularly, to a polydiacetylene-based compound, which is a polymer of a self-assembly of diacetylene monomers represented by the following Chemical Formulas 1 and 2, a composition including the same, and a chemical sensor.

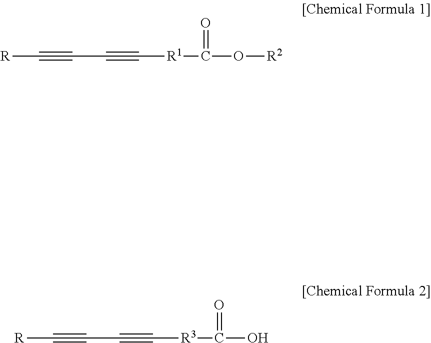

(Chemical Formulas 1 and 2 are as defined in claim 1.)

### CROSS-REFERENCE TO RELATED APPLICATION

This application claims the benefit of Korean Patent Application No. 10-2024-0071648 filed on May 31, 2024, in the Korean Intellectual Property Office, the entire disclosure of which is incorporated herein by reference for all purposes.

### BACKGROUND

### 1. Field of the Invention

One or more embodiments of the present disclosure relate to a polydiacetylene-based compound, a composition including the compound, and chemical sensors for detecting cyanide ions (CN⁻).

### 2. Description of the Related Art

Recently, with the development of science and technology, water pollution and food safety issues caused by toxic substances have received attention. Cyanide, also called potassium cyanide, is used in the production of plastics, rubber, and herbicides, used in the production of organic compounds, and also used in electroplating or gold extraction. Such cyanide causes water pollution due to factory wastewater, and results in reaching drinking water.

The cyanide is a strong toxic anion that binds to the active site of mitochondrial cytochrome oxidase to inhibit enzyme activity and interfere with electron transport, and as a result, reduces an oxidative metabolism. Poisoning symptoms of cyanide cause difficulty in breathing, headache, chest pain, vomiting, etc., when absorbed through the lungs and skin of a person. Therefore, it is a very important task to prevent human exposure to cyanide due to water pollution, and there is a need for development of a highly sensitive colorimetric sensor for detecting the cyanide.

### SUMMARY

In order to solve the above-mentioned problems, the present disclosure provides a polydiacetylene-based compound that has high selectivity for cyanide (e.g., cyanide ions) and may detect cyanide with high sensitivity by changes in optical properties (e.g., colorimetry, absorption, and fluorescence).

The present disclosure also provides a composition including a polydiacetylene-based compound according to the present disclosure and for detecting cyanide (e.g., cyanide ions).

The present disclosure also provides a chemical sensor including a polydiacetylene-based compound according to the present disclosure, having high sensitivity by changes in optical properties (e.g., colorimetry, absorption, and fluorescence), and capable of qualitative and quantitative analysis of cyanide (e.g., cyanide ions).

However, technical aspects of the present disclosure are not limited to the aforementioned purpose and other aspects which are not mentioned may be clearly understood to those skilled in the art from the following description.

According to an aspect, there is provided a polydiacetylene-based compound including a polymer of a self-assembly of diacetylene monomers, in which the diacetylene monomers may include monomers represented by the following Chemical Formulas 1 and 2.

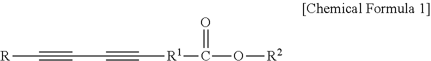

(wherein, R is an alkyl having 1 to 30 carbon atoms, R¹ is an alkylene having 1 to 30 carbon atoms, and R² is represented by the following Chemical Formula 1a,

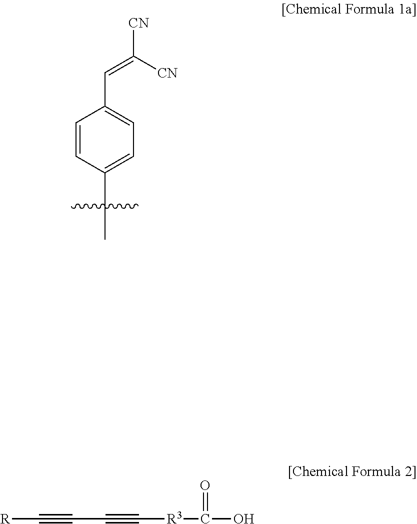

(wherein, R is an alkyl having 1 to 30 carbon atoms, and R³ is an alkylene having 1 to 30 carbon atoms.)

According to one embodiment, a molar ratio of the monomer represented by Chemical Formula 1 to the monomer represented by Chemical Formula 2 in the polydiacetylene may be 1:9 to 1:100.

According to one embodiment, the polydiacetylene-based compound may be formed by photopolymerizing a bilayer self-assembly consisting of the monomers represented by Chemical Formulas 1 and 2.

According to one embodiment, the polydiacetylene may be in the form of a self-assembled liposome.

According to one embodiment, the polydiacetylene-based compound may be polydiacetylene represented by the following Chemical Formula 3.

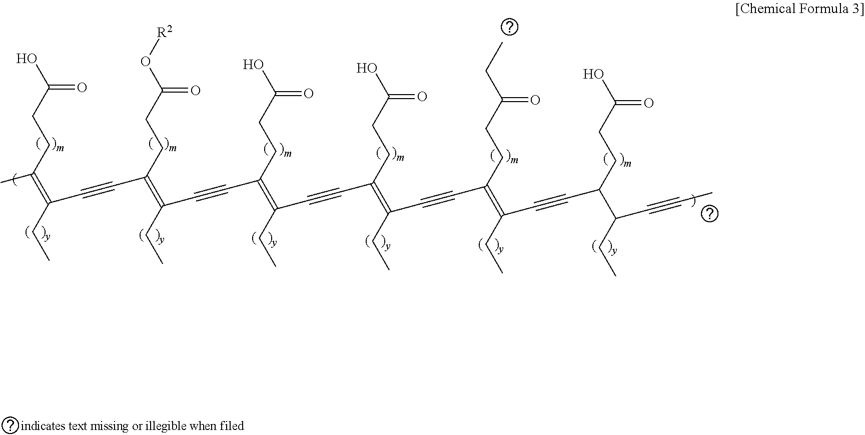

(In Chemical Formula 3, m and y are each selected from 1 to 29, and n is 1 or more.)

According to one embodiment, the composition may be used for detecting cyanide ions (CN⁻) including the polydiacetylene-based compound according to embodiments of the present disclosure.

According to one embodiment, the composition may include water, an organic solvent, or both, and the polydiacetylene-based compound may be included in an amount of more than 0 wt % to 100 wt % of the composition.

According to one embodiment, the composition may exhibit changes in optical properties when coming into contact with cyanide ions, and the changes in optical properties may be at least one of color, fluorescence wavelength, fluorescence intensity, and absorbance.

According to another aspect, there is provided a chemical sensor including a sensing unit including a polydiacetylene-based compound according to embodiments of the present disclosure.

According to one embodiment, the chemical sensor may be used for detecting cyanide ions.

According to one embodiment, the sensor may include a substrate; and a polydiacetylene-based compound coated or printed on the substrate.

According to one embodiment, the sensor may be a colorimetric sensor, and the sensor may be used for quantitative analysis, qualitative analysis, or both of cyanide ions.

According to yet another aspect, there is provided a method for detecting cyanide ions including contacting an analysis sample with a polydiacetylene-based compound according to embodiments of the present disclosure; and observing optical changes of the polydiacetylene-based compound after the contacting step.

According to one embodiment, the analysis sample may be maintained at pH of 6 to 8.

According to one embodiment, the observing step may be observing at least one or more of the color, fluorescence wavelength, absorbance, and fluorescence intensity of the polydiacetylene-based compound, or a combination thereof.

Additional aspects of embodiments will be set forth in part in the description which follows and, in part, will be apparent from the description, or may be learned by practice of the disclosure.

According to the present disclosure, it is possible to provide a polydiacetylene-based compound, a composition including the same, and a chemical sensor capable of monitoring cyanide ions with high selectivity (e.g., qualitative analysis) and sensitivity (e.g., quantitative analysis) for cyanide ions.

Further, it is possible to provide a polydiacetylene-based compound capable of real-time monitoring of water quality, a composition including the same, and a chemical sensor, which can help to prevent exposure of our bodies to a cyanide compound (e.g., cyanide ions) through drinking water and prevent damage to an ecological environment and human health caused by water pollution.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1A shows a structure, a synthesis process, and a reaction process for cyanide ions of a polydiacetylene (PDA)-based compound (PDA-CK) according to an embodiment;

FIG. 1B shows NMR data of a polydiacetylene (PDA)-based compound (PDA-CK) synthesized in Example according to an embodiment;

FIG. 2 shows photographic images showing colorimetric changes of PDA-CK in various anion environments (10 eq) according to an embodiment;

FIG. 3A shows UV-Vis absorption spectra of PDA-CK for various anions (10 eq) according to an embodiment;

FIG. 3B shows fluorescence emission spectra (λₑₓ=485 nm) of PDA-CK for various anions (10 eq) according to an embodiment;

FIG. 3C shows fluorescence emission spectra (λₑₓ=530 nm) of PDA-CK for various anions (10 eq) according to an embodiment;

FIG. 4 is a photographic image showing colorimetric changes of PDA-CK (150 μM) for increased concentrations (0 to 10 eq) of cyanide ions according to an embodiment;

FIG. 5A shows UV-Vis absorption spectra of PDA-CK for increased concentrations (0 to 10 eq) of cyanide ions according to an embodiment;

FIG. 5B shows fluorescence emission spectra (λₑₓ=485 nm) of PDA-CK for increased concentrations (0 to 10 eq) of cyanide ions according to an embodiment;

FIG. 5C shows fluorescence emission spectra (λₑₓ=530 nm) of PDA-CK for increased concentrations (0 to 10 eq) of cyanide ions according to an embodiment;

FIG. 6A shows a detection limit of PDA-CK for cyanide ions according to an embodiment;

FIG. 6B shows a colorimetric response (%) of PDA-CK for cyanide ions according to an embodiment;

FIG. 7A shows applications of PDA-CK to a paper device according to an embodiment; and

FIG. 7B shows colorimetric changes of PDA-CK for cyanide ions of a paper device according to an embodiment.

### DETAILED DESCRIPTION

Hereinafter, embodiments of the present disclosure will be described in detail with reference to the accompanying drawings. In describing the present disclosure, a detailed description of known functions or configurations will be omitted if it is determined that they unnecessarily make the gist of the present disclosure unclear. Terminologies used herein are terminologies used to properly express embodiments of the present disclosure, which may vary according to a user, an operator's intention, or customs in the art to which the present disclosure pertains. Therefore, these terminologies used herein will be defined based on the contents throughout the specification.

Throughout this specification, it will be understood that when a member is referred to as being “on” another member, it may be directly on the other member or intervening members may also be present.

Throughout the specification, when a certain part “comprises” a certain component, it will be understood to imply the inclusion of stated elements but not the exclusion of any other elements.

Hereinafter, a polydiacetylene-based compound of the present disclosure and uses thereof will be described in detail with reference to embodiments and drawings. However, the present disclosure is not limited to these embodiments and drawings.

According to one embodiment, a polydiacetylene (PDA)-based compound of the present disclosure may be a polymer of diacetylene monomers represented by the following Chemical Formulas 1 and 2. The polydiacetylene-based compound may exhibit selective colorimetric and/or fluorescence changes when binding to cyanide (i.e., cyanide ions), and may be used as a colorimetric sensor having high sensitivity and selectivity and capable of qualitative and quantitative analysis.

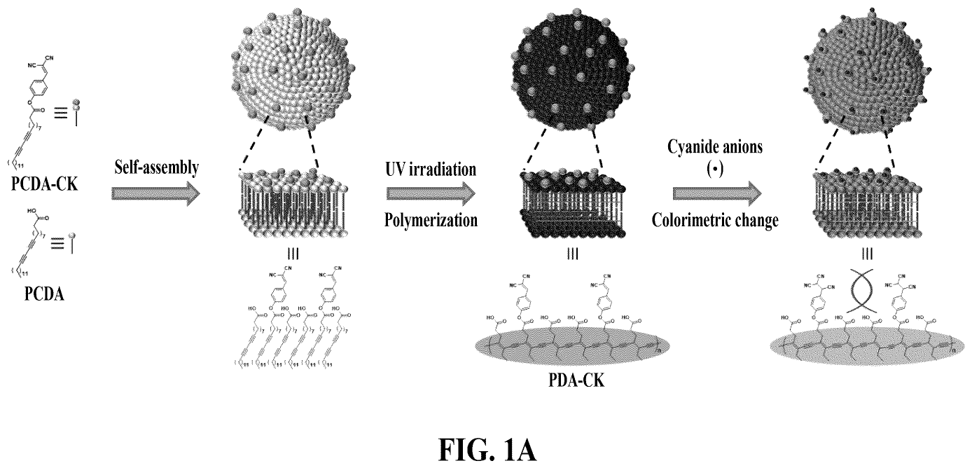

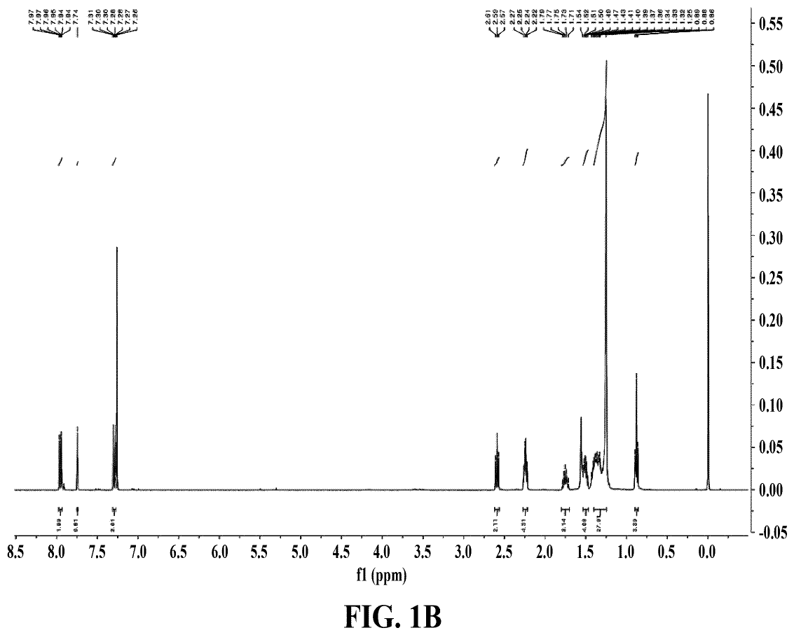

Referring to FIG. 1A, a structure of the polydiacetylene (PDA)-based compound of the present disclosure and a reaction process for cyanide ions of FIG. 1A are shown. In FIG. 1A, the polydiacetylene (PDA)-based compound (PDA-CK) of the present disclosure is a conjugated polymer and may be used as stimuli-responsive colorimetric and fluorescence chemical sensors. When aligned diacetylene monomers are irradiated with UV or plasma, the diacetylene monomers are polymerized through a 1,4-addition reaction in which “ene-yne” polymer chains are alternately formed to form polydiacetylene. The aligned diacetylene monomers are a bilayer self-assembly, and may be photopolymerized to form polydiacetylene in the form of a bilayer self-assembly. In a π-conjugation system tightly formed along a PDA polymer backbone, a well-aligned backbone structure is distorted by an external stimulus, so that a color transition and a maximum absorption band shift may occur. In addition, the polydiacetylene (PDA)-based compound has a receptor (CK) having a cyanide-based substituent with high selectivity for cyanide (cyanide ions), which may be formed by a diacetylene monomer represented by the following Chemical Formula 1. That is, as a colorimetric sensor with high selectivity for cyanide (e.g., cyanide ions), PDA-CK exhibits a colorimetric change from blue to orange for cyanide (e.g., cyanide ions), which may be confirmed by absorption and fluorescence spectrum analysis.

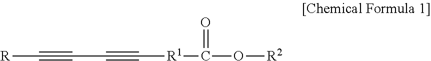

According to one embodiment, in Chemical Formula 1, R may be an alkyl having 1 to 30 carbon atoms, and R¹ may be an alkylene having 1 to 30 carbon atoms. Desirably, in Chemical Formula 1, R may be an alkyl having 3 to 20 carbon atoms; an alkyl group having 3 to 15 carbon atoms; or an alkyl group having 8 to 10 carbon atoms. In Chemical Formula 1, R¹ may be an alkylene (—(CH₂)ₙ—) having 3 to 20 carbon atoms; an alkylene having 3 to 15 carbon atoms; or an alkylene having 5 to 10 carbon atoms. The R¹ may be an alkylene group having more carbon atoms than R.

According to one embodiment, in Chemical Formula 1, R² may be represented by the following Chemical Formula 1a.

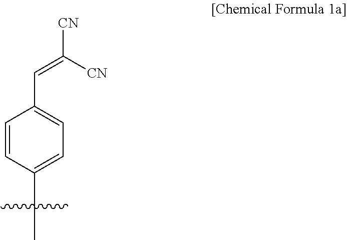 [SMILES: *c1ccc(C=C(C#N)C#N)cc1]

According to one embodiment, R² represented by Chemical Formula 1a corresponds to a receptor (CK) for detecting cyanide ions, provides high selectivity in the presence of cyanide ions, and may induce selective colorimetric and/or fluorescence changes. For example, the polydiacetylene-based compound exhibits changes in optical properties when binding to cyanide ions, and the changes in optical properties may be at least one or more of color, fluorescence wavelength, fluorescence intensity, and absorbance, or a combination thereof.

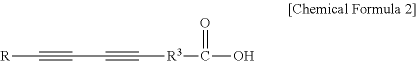

According to one embodiment, R and R³ in Chemical Formula 2 may be the same as or different from R and R¹ of Chemical Formula 1, and for example, R and R³ may be each an alkyl having 1 to 30 carbon atoms, and R³ may be an alkylene having 1 to 30 carbon atoms. Desirably, in Chemical Formula 2, R may be an alkyl having 3 to 20 carbon atoms; an alkyl group having 3 to 15 carbon atoms; or an alkyl group having 8 to 10 carbon atoms. In Chemical Formula 2, R³ may be an alkylene having 3 to 20 carbon atoms; an alkylene having 3 to 15 carbon atoms; or an alkylene having 5 to 10 carbon atoms. The R³ may be an alkylene having smaller carbon atoms than R.

According to one embodiment, the polydiacetylene-based compound according to the present disclosure may be polydiacetylene formed by forming a self-assembly of diacetylenes represented by Chemical Formulas 1 and 2, and photopolymerizing the self-assembly. The self-assembly is a double-layer structure (i.e., a double-layer self-assembly), and is in the form of a particle, a liposome, a line, a tube, a film, a thin film, a sheet, a fiber, a flake, etc., and the self-assembly has a nano- and/or micro-size, and may be obtained as a powder, a solution, a slurry, a suspension, etc.

According to one embodiment, the ratio (molar ratio) of the monomer of the diacetylene compound represented by Chemical Formula 1 to the monomer of the diacetylene compound represented by Chemical Formula 2 in the self-assembly may be 1:5 to 1:9. If the ratio is included within the above range, the self-assembly may exhibit high selectivity and sensitivity for cyanide ions.

According to one embodiment, the polydiacetylene may be formed by irradiating UV light to the diacetylene compounds represented by Chemical Formulas 1 and 2 as the monomers for 1 minute or more; for 1 minute to 30 minutes; or for 1 minute to 10 minutes and photopolymerizing the diacetylene compounds, and the polymerization process may use diacetylene compounds or self-assembled diacetylene compounds.

According to one embodiment, referring to FIG. 1A, the polydiacetylene is formed by photopolymerizing a mixture of diacetylene monomers represented by Chemical Formulas 1 and 2 or the self-assembly, and the polydiacetylene is a conjugated polymer, which may be used for the detection of cyanide (cyanide ions). More specifically, referring to FIG. 1A, a diacetylene compound (compound of Chemical Formula 1) and a diacetylene compound (compound of Chemical Formula 2) having a chelating moiety (or a receptor portion) of cyanide (cyanide ions) may be self-assembled at a certain ratio and then a conjugated polymer may be formed by photopolymerization.

According to one embodiment, the conjugated polymer may be blue and change to an orange-based color by binding to a cyanide target, i.e., cyanide ions, at pH 7.4 and pH 6.4, and may also increase in fluorescence. Such a colorimetric change may provide colorimetric and fluorescence changes due to high selectivity in reacting only with cyanide ions without reacting with other ions.

According to one embodiment, the polydiacetylene-based compound may be polydiacetylene represented by the following Chemical Formula 3.

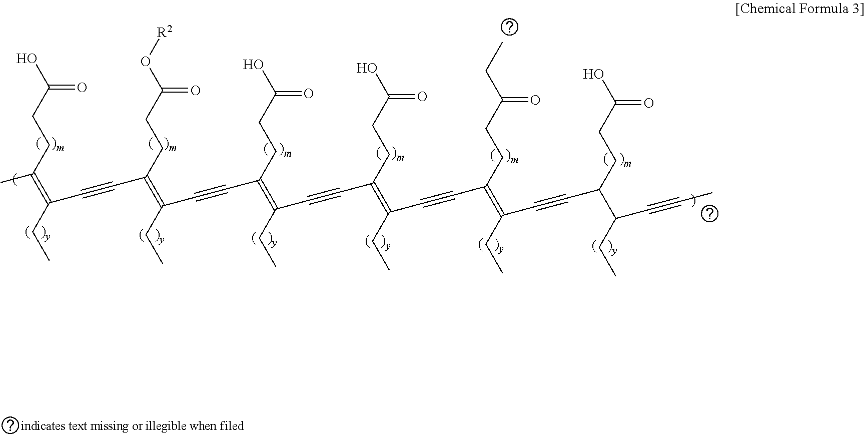

In Chemical Formula 3, m and y are each selected from 1 to 29, and n is an integer of 1 or more; 10 or more; 100 or more; or 1 to 1000, and may be set according to a molecular weight. In Chemical Formula 3, m and y are each selected from 1 to 29; or 3 to 20, and n is selected from integers of 1 or more; 1 to 10000; 1 to 5000; 10 to 1000; or 100 to 1000, and determined according to the molecular weight of the polymer. For example, the polydiacetylene-based compound may have a molecular weight (Mw or Mn) of 4,000 to 400,000; 10,000 to 400,000; 20,000 to 400,000; 10,000 to 200,000; or 10,000 to 100,000.

According to one embodiment, the polydiacetylene is a self-assembly, and the self-assembly is a bilayer structure (e.g., in the form of self-assembled liposome) and has a form such as a particle, a liposome, a line, a tube, a film, a thin film, a sheet, a fiber, a flake, etc., and the self-assembly has a nano- and/or micro-size and may be obtained as a powder, a solution, a slurry, a suspension, etc.

According to one embodiment, the present disclosure provides a composition including a compound according to the present disclosure, wherein the compound includes a polydiacetylene-based compound of the present disclosure, and the composition includes water as a solvent, and may further include an organic solvent such as an alcohol having 1 to 4 carbon atoms, if necessary.

According to one embodiment, the compound may be included in an amount of more than 0 wt % to 100 wt % or less (or less than); 90 wt % or less; 50 wt % or less; 10 wt % or less; 1 wt % or less; 0.1 wt % or less; 0.01 wt % or less; or 0.001 wt % or less of the composition.

According to one embodiment, the compound may be included at a concentration of 1×10⁻⁵ M (mol) or more, desirably 1×10⁻⁵ M (mol) to 1×10⁻² M (mol), and more desirably 1×10⁻⁴ M (mol) to 1×10⁻² M (mol) of the composition, and if the concentration is less than 10⁻⁵ M (mol), it may not be easy to observe the detection of cyanide due to the weak color change, absorbance, fluorescence intensity, etc. of the compound.

According to one embodiment, the composition is maintained at pH 6 to 8; pH 6.2 to 7.5; pH 6.4 to 7.4 or pH 6.8 to 7.4, which may be achieved with a buffer solution.

According to one embodiment, the detection limit of cyanide (e.g., cyanide ions) in the composition may be 10×10⁻⁵ to 17×10⁻⁵; or 12.2×10⁻⁵ to 16.4×10⁻⁵ M (mol), and for example, 16.4×10⁻⁵ at pH 7.4 and 12.2×10⁻⁵ at pH 6.8. The detection concentration of the cyanide may be within 100 times the detection limit.

According to one embodiment, the composition is used for detecting cyanide (e.g., cyanide ions), and the composition changes in optical properties when coming into contact with cyanide, and for example, a change in at least one of color, fluorescence wavelength, fluorescence intensity, and absorbance may be observed. In addition, the color transition may be used to confirm the presence of cyanide by observing a change in color, etc. with the naked eye without special equipment, and further, may be used for qualitative and/or quantitative analysis of cyanide using a UV-Vis spectrometer, a fluorescence spectrometer, etc.

According to one embodiment, the present disclosure may provide a product and a device for sensing and/or detecting cyanide including a compound or composition according to the present disclosure. According to one embodiment, the product and the device may be a chemical sensor or a kit.

According to one embodiment, the present disclosure may provide a chemical sensor including a sensing unit including a compound or composition according to the present disclosure.

According to one embodiment, the colorimetric sensor may be a colorimetric sensor used for sensing and/or detecting cyanide and capable of quantitative and/or qualitative analysis of cyanide.

According to one embodiment, the compound or composition may include one or at least two of polydiacetylene (PDA)-based compounds according to the present disclosure.

According to one embodiment, the compound or composition may be applied as a powder, a gel, an emulsion, or a liquid, or as a molded article, or may be coated or impregnated on a support such as an analysis chip, an electric circuit, a fiber, pulp, a polymer film, a glass substrate, etc., to be applied to the chemical sensor.

According to one embodiment, the chemical sensor may visually, electrically, and/or optically sense and measure a color change of the compound or composition to perform qualitative and/or quantitative analysis. The chemical sensor may further be equipped with a commonly used analysis device, without departing from the scope and purpose of the present disclosure, and is not specifically mentioned in the present disclosure.

According to one embodiment, the chemical sensor may include a substrate; and a sensing unit having a region on the substrate in which a polydiacetylene (PDA)-based compound or composition of the present disclosure is included. The substrate may be a polymer, pulp, fiber, glass, etc., and the substrate may be paper, a film, a sheet, a bead, a fabric, a nonwoven fabric, etc. The sensing unit may be formed by coating, impregnation, or printing. For example, the sensing unit may be a paper sensor.

According to one embodiment, the sensor may further include a commonly used configuration, without departing from the scope of the present disclosure, and is not specifically mentioned in the present application.

According to one embodiment, there may be provided a kit for detecting cyanide including a compound or composition according to the present disclosure. The kit may be portable so as to be used in a laboratory or in the field. The kit may include the compound or composition in the same manner as mentioned in the chemical sensor.

According to one embodiment, the kit may visually, electrically, and/or optically sense and measure a color change of the compound or composition to perform qualitative and/or quantitative analysis.

According to one embodiment, the kit may further include an absorbance or fluorescence intensity meter, and the meter may be integrated with the kit or configured separately from the kit.

According to one embodiment, the configuration of the kit may further include a configuration commonly used in the kit, without departing from the scope of the present disclosure, and is not specifically mentioned in the present application.

According to one embodiment, the present disclosure relates to an analysis method for a detection target using the compound or composition according to the present disclosure, wherein the analysis method may include contacting an analysis sample; and observing optical changes. The analysis method is a method for sensing and/or detecting cyanide (e.g., cyanide ions) and may be used for quantitative and/or qualitative analysis of the cyanide (e.g., cyanide ions).

According to one embodiment, in the analysis method, the contacting of the analysis sample is a step of contacting the analysis sample with the compound or composition according to the present disclosure.

According to one embodiment, the compound or composition may include a polydiacetylene (PDA)-based compound, which is a polymer of a self-assembly of diacetylene monomers represented by Chemical Formulas 1 and 2 according to the present disclosure.

According to one embodiment, the analysis sample may be a liquid, gas, aerosol, gel or powder.

According to one embodiment, the observing of the optical change is a step of observing the optical change of the compound or composition after the contacting step. The quantitative and qualitative analysis of the detection target, i.e., cyanide, may be performed.

According to one embodiment, the observing step may be observing at least one of the color, fluorescence wavelength, absorbance, and fluorescence intensity of the compound or composition. That is, the optical change is a change in color, absorbance, or fluorescence intensity, and such an optical change may be used for quantitative and qualitative analysis using the naked eye, a UV-Vis spectrometer, a fluorescence photometer, etc. For example, when a detection target, cyanide (e.g., cyanide ions), for example, cyanide ions, cyanide, and/or a cyanide compound is present in the analysis sample, the color of the composition changes from blue to an orange-based color, so that the presence of cyanide may be detected, and 540 nm absorption bands may increase in the measurement of absorbance according to a UV-Vis spectrometer. In addition, since the absorption band, the intensity of fluorescence emission, and the like change depending on a concentration of cyanide, quantitative analysis may be performed.

According to one embodiment, in the analysis method, the detection limit of the cyanide may be 10×10⁻⁵ to 17×10⁻⁵; or 12.2×10⁻⁵ to 16.4×10⁻⁵ M (mol), for example, 16.4×10⁻⁵ at pH 7.4 and 12.2×10⁻⁵ at pH 6.8. The analysis sample and/or the composition are maintained at pH 6 to 8; pH 6.2 to 7.5; pH 6.4 to 7.4; approximately pH 6.8 or approximately 7.4, which may be achieved with a buffer solution. The buffer solution may be applied within the technical field of the present disclosure and applied without limitation, without departing from the purpose of the present disclosure.

### Examples

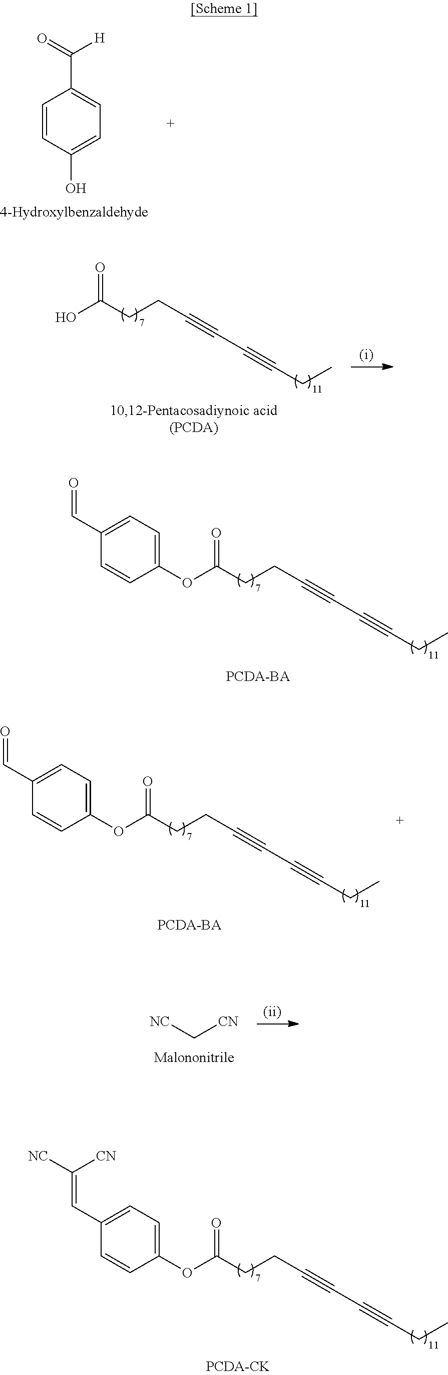

### Synthesis Example

Synthesis was performed according to Scheme 1. PCDA-BA was synthesized as follows.

PCDA (10,12-Pentacosadiynoic acid) (0.5619 g, 1.5 mmol), 4-Hydroxybenzaldehyde (0.2442 g, 2 mmol), HBTU (2-(1H-Benzotriazole-1-yl)-1,1,3,3-tetramethyluronium hexafluorophosphate Novabiochem; 0.7585 g, 2 mmol), DMAP (4-dimethylamionpyridine; catalytic), and N,N-diisopropylethylamine (0.1939 g, 3 mmol) were stirred in DCM (50 mL) at room temperature for 12 hour. After the reaction was completed, the mixture was washed with distilled water and purified by column chromatography using DCM as a developing solvent to obtain PCDA-BA in 94% yield. 1H NMR (400 MHz, chloroform-d, 8) 9.99 (s, 1H), 7.94-7.90 (dt, J=8.6, 2 Hz, 2H), 7.29-7.25 (dt, J=8.6, 2 Hz, 2H), 2.60-2.57 (t, J=7.6 Hz, 2H), 2.27-2.22 (m, 4H) 1.79-1.72 (quint, J=7.6 Hz, 2H), 1.56-1.47 (m, 4H), 1.43-1.25 (m, 26H), 0.89-0.86 (t, J=6.8 Hz, 3H). ESI HRMS m/z=479.3513 [M+H]+, calc. for C32H46O3=478.34.

The PCDA-CK was prepared by refluxing a mixture of PCDA-BA (0.4935 g, 1.03 mmol) and malononitrile (0.1238 g, 1.87 mmol) in ethanol for 6 hours. After the reaction was completed, the solvent of the mixture was removed and the mixture was purified by column chromatography using Hexane/DCM (v/v, 1/1) as a developing solvent to obtain PCDA-CK in yield 70%. 1H NMR (400 MHz, chloroform-d, 8) 7.97-7.94 (dt, J=8.6, 2.2 Hz, 2H), 7.74 (s, 1H), 7.31-7.27 (dt, J=8.6, 2.2 Hz, 2H), 2.61-2.57 (t, J=7.6 Hz, 2H), 2.27-2.22 (m, 4H), 1.79-1.71 (quint, J=7.6 Hz, 2H), 1.54-1.47 (m, 4H), 1.43-1.25 (m, 26H), 0.89-0.86 (t, J=6.8 Hz, 3H) (FIG. 1B). ESI HRMS m/z=527.3629 [M+H]+, calc. for C35H46N2O2=526.36.

### Preparation of Diacetylene Self-Assembly and Photopolymerization Thereof

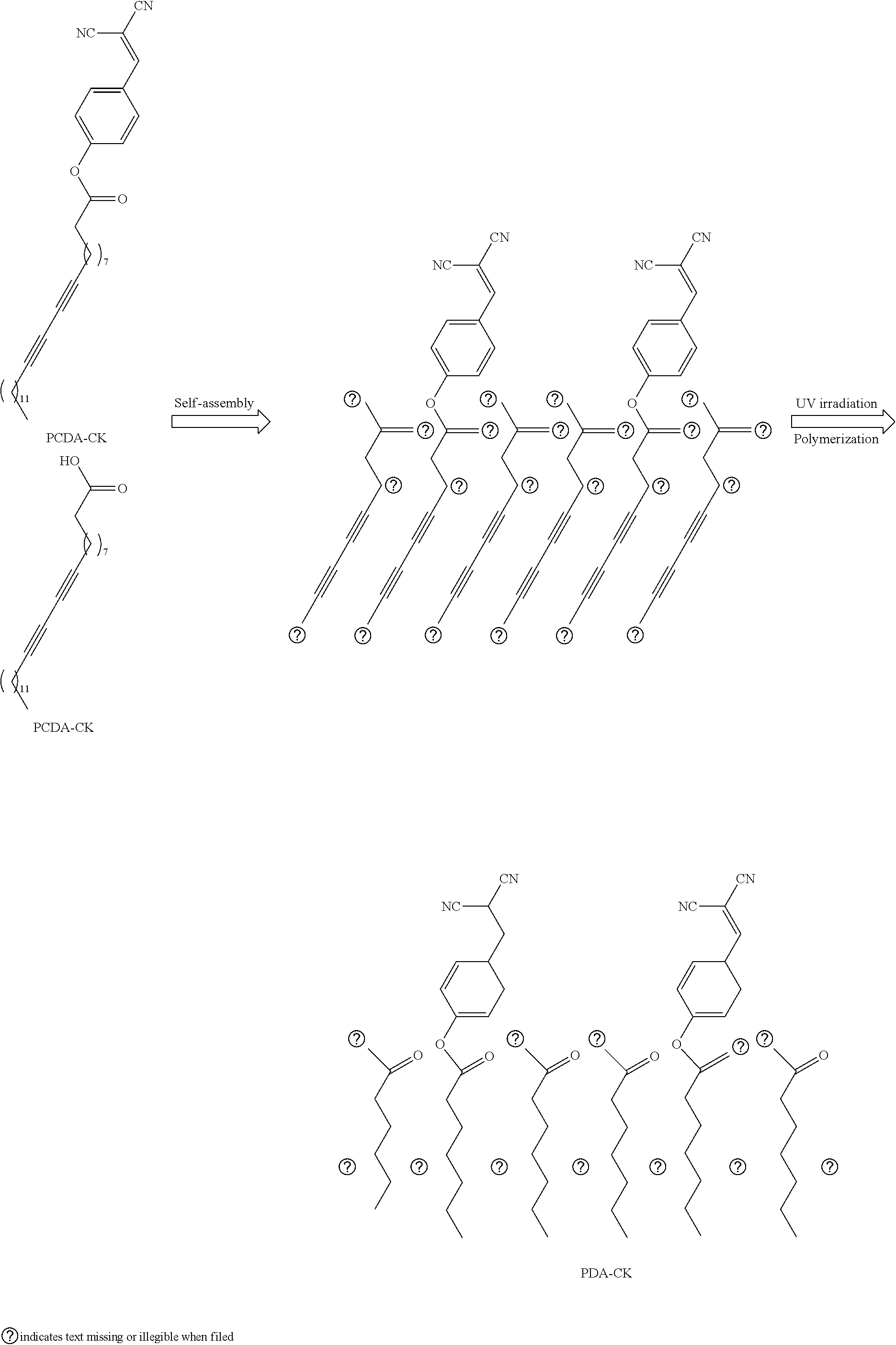

Referring to Scheme 2 and FIG. 1A, a diacetylene self-assembly and a photopolymerization thereof were prepared.

A lipid solution in an aqueous solution was prepared by the following method. A monomer (PCDA-CK) (1.1 mg, 1 eq) and PCDA (6.7 mg, 9 eq) at a 1:9 ratio were dissolved in 1 mL of DMSO. The mixture was sonicated in 19 mL of distilled water at 80° C., and after sonication for 1 hour, lipid aggregates were removed by passing through a 600 nm syringe filter. The filtrate was incubated overnight at 4° C. A colorimetric sensor PDA-CK was fabricated by polymerizing the solution at room temperature by irradiating the PCDA-CK solution with 245 nm UV light (1 mW/cm²) for 10 minutes.

### Detection Characteristics of Cyanide Ions

### <Selectivity of PDA-CK for Cyanide Ions>

The selectivity of PDA-CK (150 μM) for cyanide ions was investigated through colorimetric changes and UV-vis spectra in the analysis of various anions (F⁻, Cl⁻, Br⁻, I⁻, NO₃⁻, ClO₄⁻, H₂PO₄⁻, HCO₃⁻, SCN⁻, BzO⁻, AcO⁻, N₃⁻, and CN⁻).

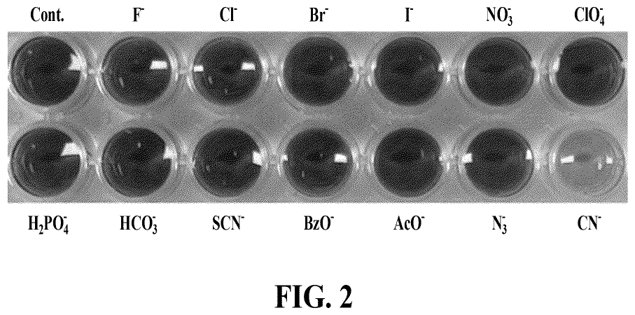

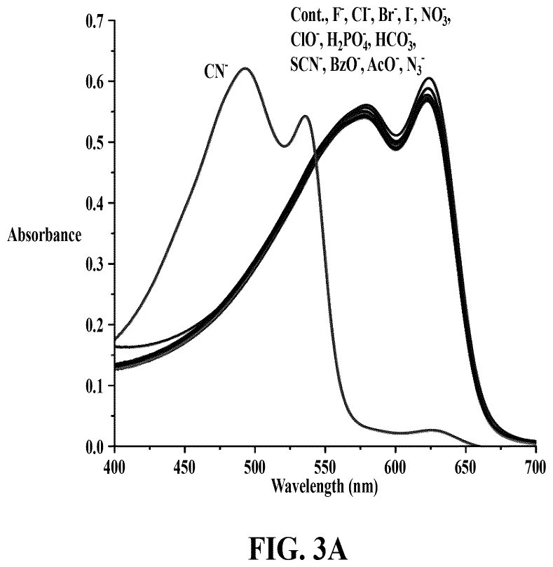

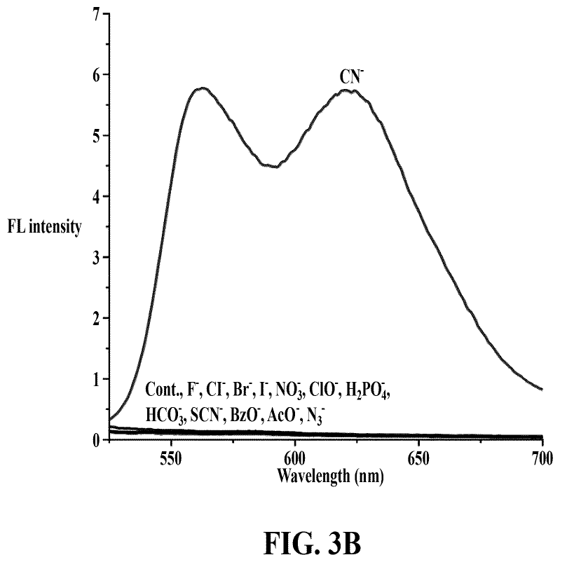

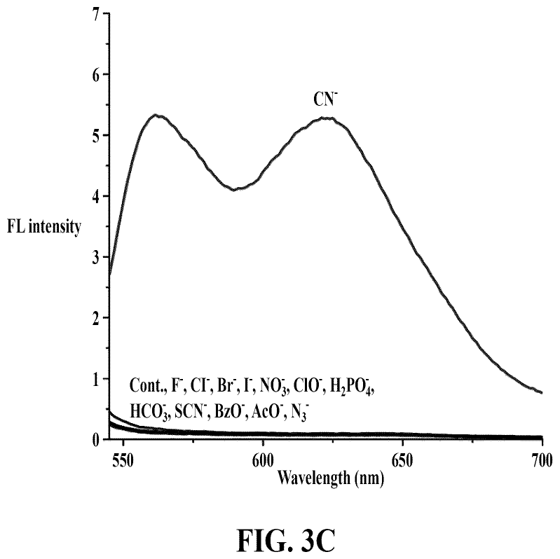

The recognition of various ions by PDA-CK was investigated through colorimetric changes, UV absorption and fluorescence emission changes in the aqueous solution (PDA-CK (150 μM)). The results were shown in FIGS. 2, 3A, 3B, and 3C.

FIG. 2 is a photographic image showing colorimetric changes of PDA-CK (150 μM) in various anion environments (10 eq). That is, according to the following Reaction Schemes 1 and 2, the PDA-CK of the present disclosure changed from blue to orange-based colors when reacting with cyanide ions, but no colorimetric change was observed in other ion environments. It may be confirmed that the PDA-CK has selectivity for cyanide ions. That is, the colorimetric change may confirm visual detection of cyanide ions without any special equipment, and the color change is also observed quickly, and the PDA-CK may exhibit high selectivity by reacting only with cyanide ions without reacting with other ions.

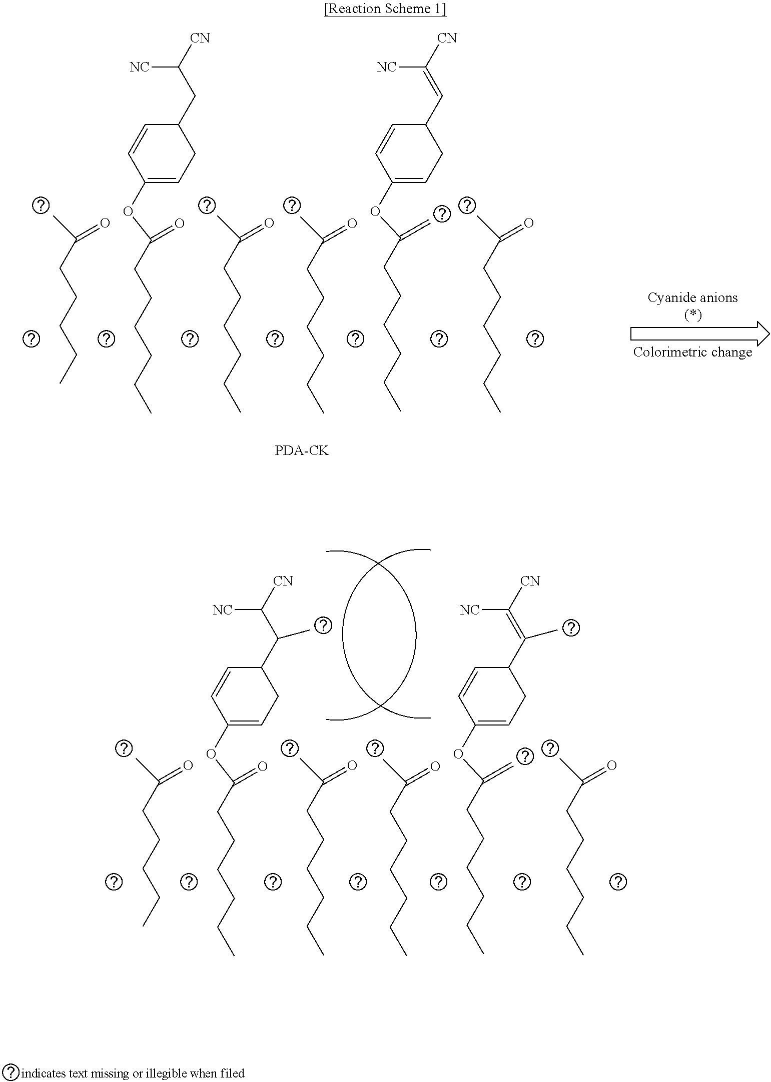

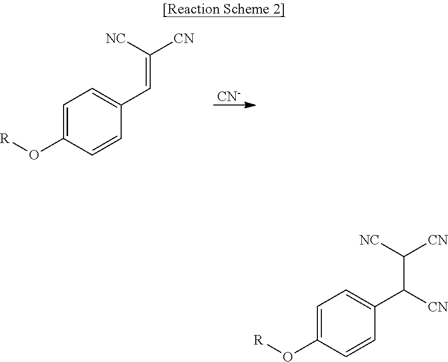

FIGS. 3A, 3B, and 3C show UV-Vis absorption spectra (FIG. 3A), fluorescence emission spectra (λₑₓ=485 nm) (FIG. 3B), and fluorescence emission spectra (λₑₓ=530 nm) (FIG. 3C) of PDA-CK for various anions (10 eq). In FIG. 3A, FIG. 3B and FIG. 3C, it may be confirmed that PDA-CK has optical properties (i.e., absorption and emission intensities) that are distinguished from other anions in a cyanide ion environment. Through this, the PDA-CK of the present disclosure may be used for qualitative analysis of cyanide ions.

### <Sensitivity of PDA-CK for Cyanide Ions>

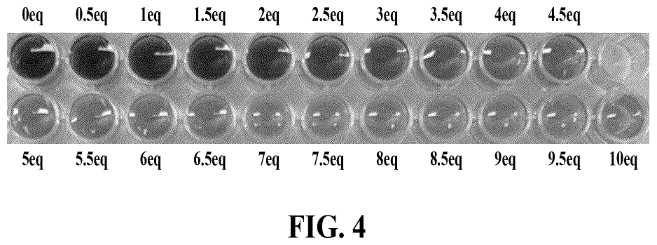

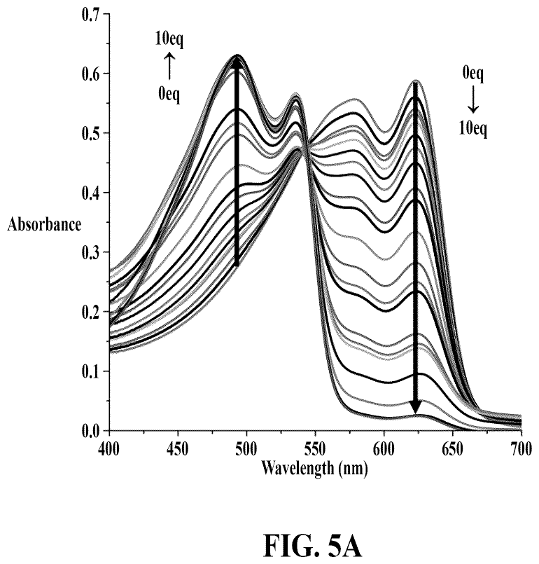

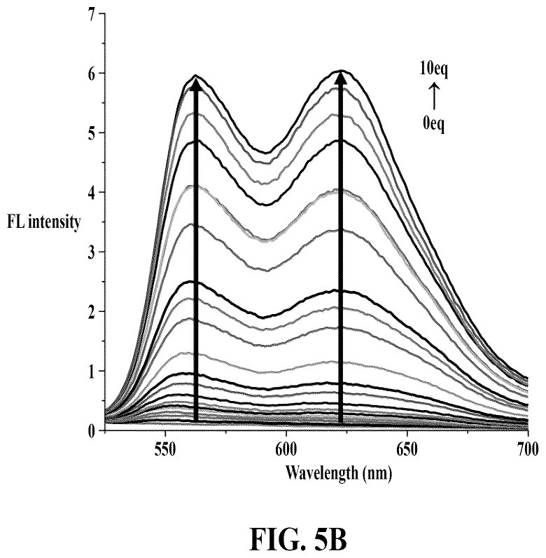

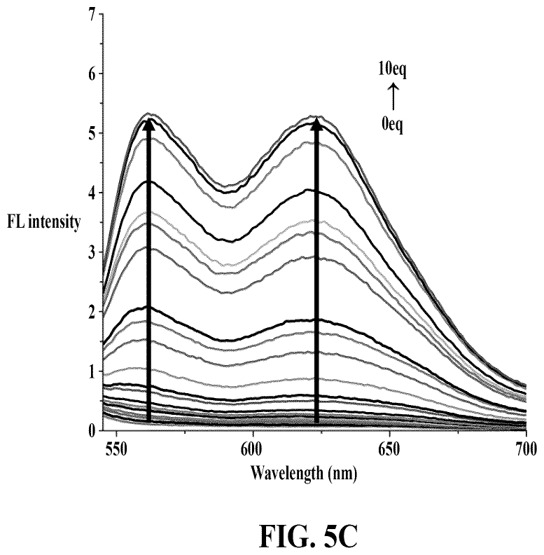

The sensitivity of PDA-CK for cyanide ions was investigated through colorimetric changes and UV-vis spectra depending on a concentration of cyanide ions (CN⁻). The results were shown in FIGS. 4, 5A, 5B, and 5C.

FIG. 4 is a photographic image showing colorimetric changes of PDA-CK (150 μM) for increased concentrations (0 to 10 eq) of cyanide ions. In FIG. 4, a difference in the colorimetric change may be confirmed with the naked eye depending on a concentration of cyanide ions.

FIGS. 5A, 5B, and 5C show UV-Vis absorption spectra (FIG. 5A), fluorescence emission spectra (λₑₓ=485 nm) (FIG. 5B), and fluorescence emission spectra (λₑₓ=530 nm) (FIG. 5C) of PDA-CK for increased concentrations (0 to 10 eq) of cyanide ions. In FIGS. 5A, 5B, and 5C, it may be confirmed that the absorption and emission intensities change with the concentration of cyanide ions. Through this, the PDA-CK of the present disclosure may be used for quantitative analysis of cyanide ions.

### <Detection Limit (LOD) and Colorimetric Response of PDA-CK for Cyanide Ions>

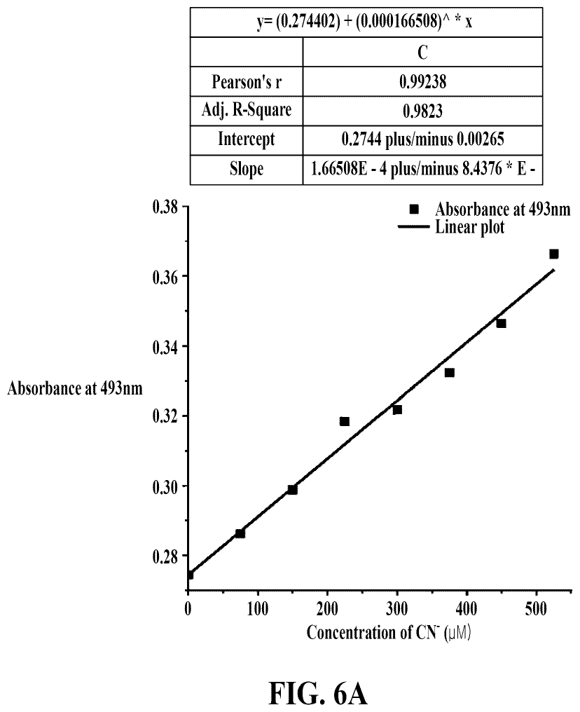

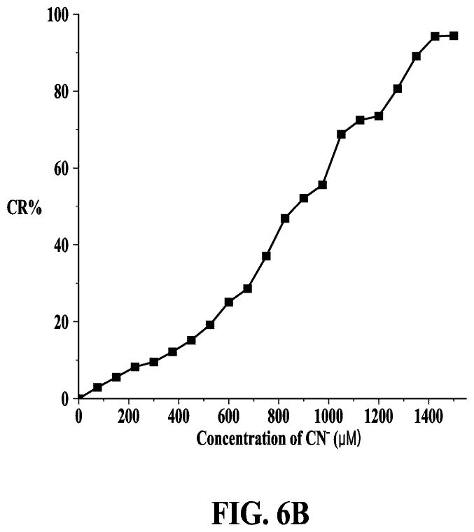

The detection limit (LOD) and colorimetric response of PDA-CK for cyanide ions were evaluated and shown in FIGS. 6A and 6B.

FIGS. 6A and 6B show the detection limit (FIG. 6A) and the colorimetric response (%) (FIG. 6B) of PDA-CK. The detection limit in FIG. 6A was calculated using the following equation, and the result was LOD (Limit of Detection)=0.66 μM.

$$LOD = 3 \times δ \div S$$

(wherein, δ represents a standard deviation of the blank measurement, and S represents a slope of intensity vs. sample concentration curve.)

The results of the colorimetric response in FIG. 6B were shown in Table 1.

TABLE 1

| [CN⁻] | 600 μM | 900 μM | 1500 μM |
|---|---|---|---|
| (μM) | (4 eq) | (6 eq) | (10 eq) |
| CR % | 25.1% | 52.2% | 94.4% |

In FIG. 6B and Table 1, it may be confirmed that the colorimetric response of the PDA-CK of the present disclosure increases as the concentration of cyanide ions increases.

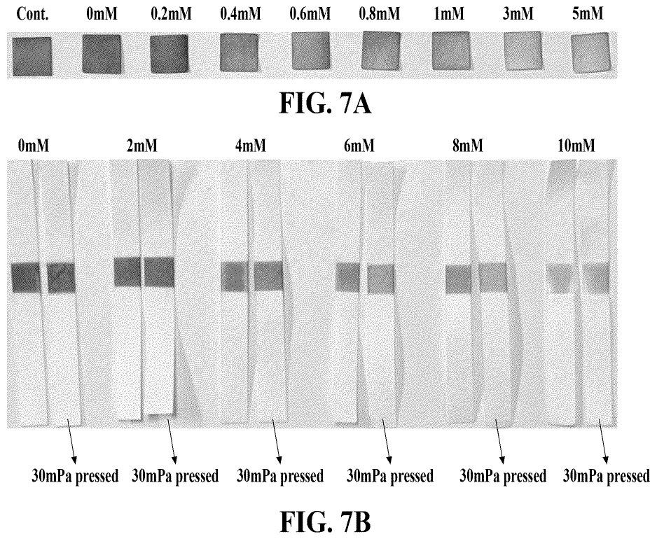

FIGS. 7A and 7B show applications of PDA-CK of the present disclosure to a paper device. FIG. 7A shows a paper device immersed in each CN-solution for 1 hour, and FIG. 7B shows a paper device flowing in each CN-solution for 15 minutes. As shown in FIGS. 7A and 7B, a PDA-CK printing (or adsorption) area is formed on the paper, and the colorimetric change may be visually confirmed in a cyanide ion environment. Such a paper device may be used as a colorimetric sensor device.

The present disclosure may provide a PDA-CK as a polydiacetylene (PDA) colorimetric sensor with high selectivity and high sensitivity for cyanide by combining 4-hydroxybenzaldehyde and malononitrile to synthesize a receptor (CK) with high selectivity for cyanide, binding the synthesized receptor to PCDA as a monomer of a PDA sensor, and then polymerizing the mixture. The PDA-CK of the present disclosure may monitor cyanide ions with excellent selectivity (e.g., qualitative analysis) and sensitivity (e.g., quantitative analysis) for cyanide ions. The PDA-CK may rapidly detect cyanide ions with the naked eye and also easily detect the cyanide ions by absorption spectrum analysis. This may be applied to real-time water quality environment monitoring.

As described above, although the embodiments have been described by the restricted embodiments and the drawings, various modifications and variations may be made from the above description by those skilled in the art. For example, even if the described techniques are performed in a different order from the described method, and/or components described above are coupled or combined in a different form from the described method, or replaced or substituted by other components or equivalents, an appropriate result may be achieved. Therefore, other implementations, other embodiments, and equivalents to the appended claims fall within the scope of the claims to be described below.

What is claimed is:

1. A polydiacetylene-based compound which is a polymer of a self-assembly of diacetylene monomers,
wherein the diacetylene monomers comprise monomers represented by the following Chemical Formulas 1 and 2: 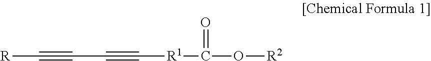
(wherein, R is an alkyl having 1 to 30 carbon atoms, R¹ is an alkylene having 1 to 30 carbon atoms, and R² is represented by the following Chemical Formula 1a, 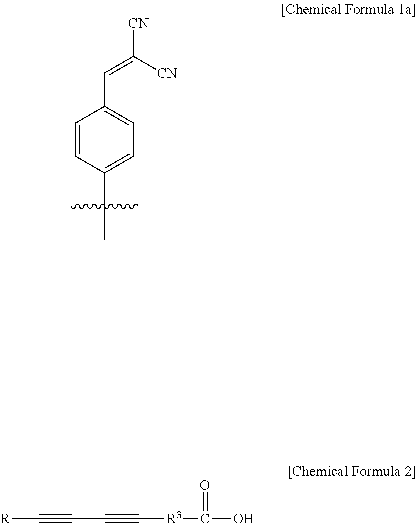
(wherein, R is an alkyl having 1 to 30 carbon atoms, and R³ is an alkylene having 1 to 30 carbon atoms).

2. The polydiacetylene-based compound of claim 1, wherein a molar ratio of the monomer represented by Chemical Formula 1 to the monomer represented by the Chemical Formula 2 in the polydiacetylene is 1:9 to 1:100.

3. The polydiacetylene-based compound of claim 1, wherein the polydiacetylene-based compound is formed by photopolymerizing a bilayer self-assembly consisting of monomers represented by Chemical Formulas 1 and 2.

4. The polydiacetylene-based compound of claim 1, wherein the polydiacetylene is in the form of a self-assembled liposome.

5. The polydiacetylene-based compound of claim 1, wherein the polydiacetylene-based compound is polydiacetylene represented by the following Chemical Formula 3: 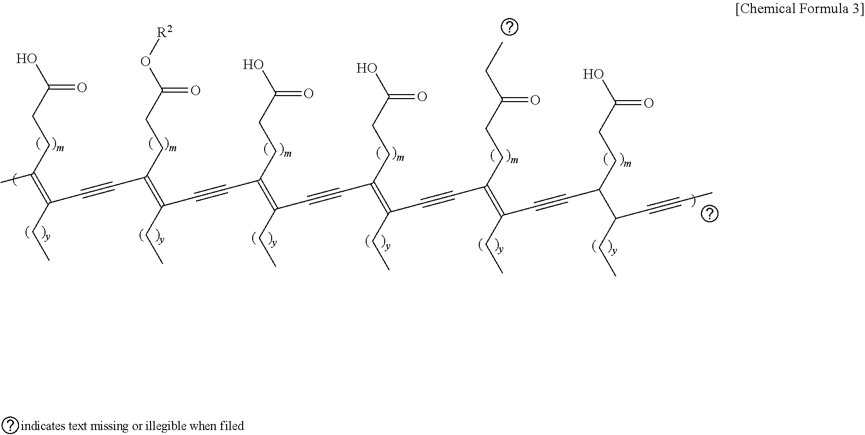
(In Chemical Formula 3, m and y are each selected from 1 to 29, and n is 1 or more).

6. A composition comprising the polydiacetylene-based compound of claim 4, used for detecting cyanide ions (CN⁻).

7. The composition of claim 6, wherein the composition comprises water, an organic solvent, or both, and
the polydiacetylene-based compound is comprised in an amount of more than 0 wt % to 100 wt % of the composition.

8. The composition of claim 6, wherein the composition exhibits changes in optical properties when coming into contact with cyanide ions, and
the changes in optical properties are at least one of color, fluorescence wavelength, fluorescence intensity, and absorbance.

9. A chemical sensor comprising a sensing unit comprising the polydiacetylene-based compound of claim 1.

10. The chemical sensor of claim 9, wherein the chemical sensor is used for detecting cyanide ions.

11. The chemical sensor of claim 9, wherein the sensor comprises a substrate; and a polydiacetylene-based compound coated or printed on the substrate.

12. The chemical sensor of claim 9, wherein the sensor is a colorimetric sensor, and
the sensor is used for quantitative analysis, qualitative analysis, or both of cyanide ions.

13. A method for detecting cyanide ions comprising:
contacting an analysis sample with the polydiacetylene-based compound of claim 1; and
observing optical changes of the polydiacetylene-based compound after the contacting.

14. The method for detecting cyanide ions of claim 13, wherein the analysis sample is maintained at pH 6 to 8.

15. The method for detecting cyanide ions of claim 13, wherein the observing is observing at least one or more of the color, fluorescence wavelength, absorbance, and fluorescence intensity of the polydiacetylene-based compound, or a combination thereof.
